(19) United States
(12) Patent Application Publication
(10) Pub. No.: US 2020/0037923 A1
(43) Pub. Date: Feb. 6, 2020

(54) SYSTEM FOR MONITORING SURGICAL SITE AND METHOD FOR MONITORING SURGICAL SITE

(71) Applicant: UNIVERSITY OF TSUKUBA, Tsukuba-shi, Ibaraki (JP)

(72) Inventors: Jian LU, Ibaraki (JP); Ryutaro MAEDA, Ibaraki (JP); Lan ZHANG, Ibaraki (JP); Hiroshi HIROSHIMA, Ibaraki (JP); Nobuhiro OHKOHCHI, Ibaraki (JP); Masanao KURATA, Ibaraki (JP); Youichi OKUDA, Ibaraki (JP)

(73) Assignee: University of Tsukuba, Tsukuba-shi, Ibaraki (JP)

(21) Appl. No.: 16/337,733

(22) PCT Filed: Jul. 20, 2017
(86) PCT No.: PCT/JP2017/026242

§ 371 Date: Mar. 28, 2019

(30) Foreign Application Priority Data
Sep. 30, 2016 (JP) 2016-192395

(51) Int. Cl. A61B 5/06; A61B 5/00

(52) U.S. Cl. CPC A61B 5/066; A61B 2562/085; A61B 5/7425; A61B 5/062

(57) ABSTRACT

The purpose of the present invention is to enable, even in an endoscopic operation, an operation upon a specific operation site while monitoring the overall surgical site, such as an organ. An artificial magnetic field is generated such that a surgical site of a patient is positioned within a generated three-dimensional artificial magnetic field space. A plurality of magnetic sensors are respectively mounted in a plurality of predetermined locations of the surgical site of the patient. Data of a 3-D image of the surgical site and position information of the mounting locations of the magnetic sensors with respect to the surgical site are stored in a storage unit as position information of the mounting locations with respect to the 3-D image of the surgical site.

### TECHNICAL FIELD OF THE INVENTION

Our invention relates to a system and a method for monitoring surgical site, suitable for monitoring surgical site in a minimally invasive surgical operation.

### BACKGROUND ART OF THE INVENTION

To perform a surgical operation properly in a short time, technologies such as sensor and TV monitor are often secondarily used recently. Patent document 1 (U.S. Pat. No. 6,633,773B1) discloses an invention about an operation of reorganization of surface of organ such as heart. In Patent document 1, a monitor displays a position of reference magnetic sensor positioned at a certain surgical site of organ of the patient while magnetic field is generated by electromagnet (coil) provided at the bottom of operating table.

In Patent document 1, the monitor also displays a position of catheter tip detected by a magnetic sensor attached thereto, when the catheter is inserted into an organ inside the body of patient. Then, the surgeon performs a necessary operation by inserting the catheter into the organ according to the position of the reference magnetic sensor, as checking the positions of the reference magnetic sensor and the magnetic sensor on the catheter tip on the monitor.

The invention of Patent document 1 would be very helpful for the surgeon to insert the catheter to a target organ properly relatively in a short time according to the position of the reference magnetic sensor.

### PRIOR ART DOCUMENTS

### Patent Documents

Patent document 1: U.S. Pat. No. 6,633,773B1

### SUMMARY OF THE INVENTION

### Problems to be Solved by the Invention

Recent surgical operation applies a minimally invasive surgical operation such as endoscopic operation to reduce the burden on patient. The operation is performed with surgical implements such as surgeon's knife while the monitor displays sites visible with an endoscope such as laparoscope. The above-described technologies of Patent document 1 can be applied to the endoscopic operation, so that the surgical implements for the endoscopic operation can be inserted to a target site to be operated more properly and quickly.

However, a narrow field of vision of the endoscope makes it difficult to grasp a whole organ in the operation. Further, technologies of grasping a whole organ in the endoscopic operation or the like have not yet developed sufficiently.

Accordingly, it could be helpful to grasp a whole surgical site such as organ to be operated during an operation of a specific operation site.

### Means for Solving the Problems

To solve the above-described object, our invention is a system for monitoring a surgical site, comprising: an artificial magnetic field generation device to generate a 3-D artificial magnetic field space in which a surgical site of a patient is disposed; a plurality of magnetic sensors attached to attachment places in the surgical site of the patient to detect a position of the attachment place in the artificial magnetic field; and a display control device to obtain output information output from the magnetic sensors as identifiable to each other, wherein the display control unit comprises: a monitor unit provided with a display screen; a memory unit to store a 3-D image data of the surgical site and a positional information of the attachment place with respect to a 3-D image of the surgical site; and a control unit to display the 3-D image, which corresponds to a present condition of the surgical site within the artificial magnetic field space, on the display screen of the monitor unit based on the 3-D image data stored in the memory unit, the positional information of the attachment place of the magnetic sensor with respect to the 3-D image of the surgical site, and the output information output from the plurality of magnetic sensors.

It is preferable that the control unit of the display control unit changes the 3-D image of the surgical site displayed on the display screen according to a change of the positional information of the attachment place of the magnetic sensor in the artificial magnetic field space.

In our surgical site monitoring system, a plurality of magnetic sensors are attached to a predetermined space (position) of the surgical site of a patient. The artificial magnetic field generation device generates artificial magnetic field to dispose the surgical sites of the patient within an artificial magnetic field space generated. Because the pair of intensity and direction of magnetic field at each position (3-D position) in the artificial magnetic field space is unique to the position, the position corresponding to values of intensity and direction of the magnetic field detected by a magnetic sensor can be identified in the artificial magnetic field space.

In our surgical site monitoring system, the memory unit preliminarily stores 3-D image data of the surgical site and positional information of the attachment place of magnetic sensors in the surgical site of the patient with respect to a 3-D image of the surgical site.

The display control device displays the 3-D image, which corresponds to a present condition of the surgical site within the artificial magnetic field space, on the display screen of the monitor unit by using the 3-D image data of the surgical site of patient stored in the memory unit, the positional information of the attachment place of the magnetic sensor with respect to the 3-D image of the surgical site, and the output information of the plurality of magnetic sensors. Namely, the display control unit detects each position of magnetic sensors in the artificial magnetic field space from the output information of magnetic sensors and displays the 3-D image on the display screen so that the detected position is the same as a position of the attachment place of the magnetic sensor of the 3-D image of the surgical site of patient. Thus, the monitor unit displays the 3-D image of the surgical site of patient corresponding to the present condition of the surgical site of real patient on the display screen.

Therefore, the surgeon can easily recognize the direction or the like of the surgical site of supine patient on the operating table by looking at the 3-D image displayed on the display screen of the monitor unit, so that the operation can be performed properly and quickly with reference thereto.

It is preferable that the control unit of the display control unit changes the 3-D image of the surgical site displayed on the display screen according to a change of the positional information of the attachment place of the magnetic sensor in the artificial magnetic field space. With this configuration, the surgeon can properly grasp a possible move of the supine patient on the operating table corresponding to the change of 3-D image of the surgical site displayed on the display screen of the monitor.

### Effect According to the Invention

Our invention makes it possible to display 3-D image of the surgical site of the patient upon a display screen of a monitor unit so as to correspond to the state of the presence of the surgical site within the artificial magnetic field space, so that the surgeon can perform a surgical operation properly and quickly by checking it.

Further, our invention makes it possible for the surgeon to properly grasp a possible move of the supine patient on the operating table corresponding to the change of 3-D image of the surgical site displayed on the display screen of the monitor.

### BRIEF EXPLANATION OF THE DRAWINGS

FIG. 1 is an overview showing an example of our surgical site monitoring system.

FIG. 2 is an explanation view of main part of an example of our surgical site monitoring system.

FIG. 3 shows an example of position of magnetic sensor attached to our surgical site monitoring system.

FIG. 4 is an explanation view of magnetic sensor used in an example of our surgical site monitoring system.

FIG. 5 is a block diagram showing a hardware configuration example of control device of display control device of our surgical site monitoring system.

FIG. 6 is an explanation view of calibration processing of magnetic sensor used in an example of our surgical site monitoring system.

FIG. 7 is an explanation view of an improved example of our surgical site monitoring system.

FIG. 8 is another explanation view of an improved example of our surgical site monitoring system.

FIG. 9 is yet another explanation view of an improved example of our surgical site monitoring system.

FIG. 10 shows an overview of another example of our surgical site monitoring system.

### EMBODIMENTS FOR CARRYING OUT THE INVENTION

Hereinafter, examples of our surgical site monitoring system and our surgical site monitoring method will be explained with reference to figures, specifically for a case that the surgical site is liver.

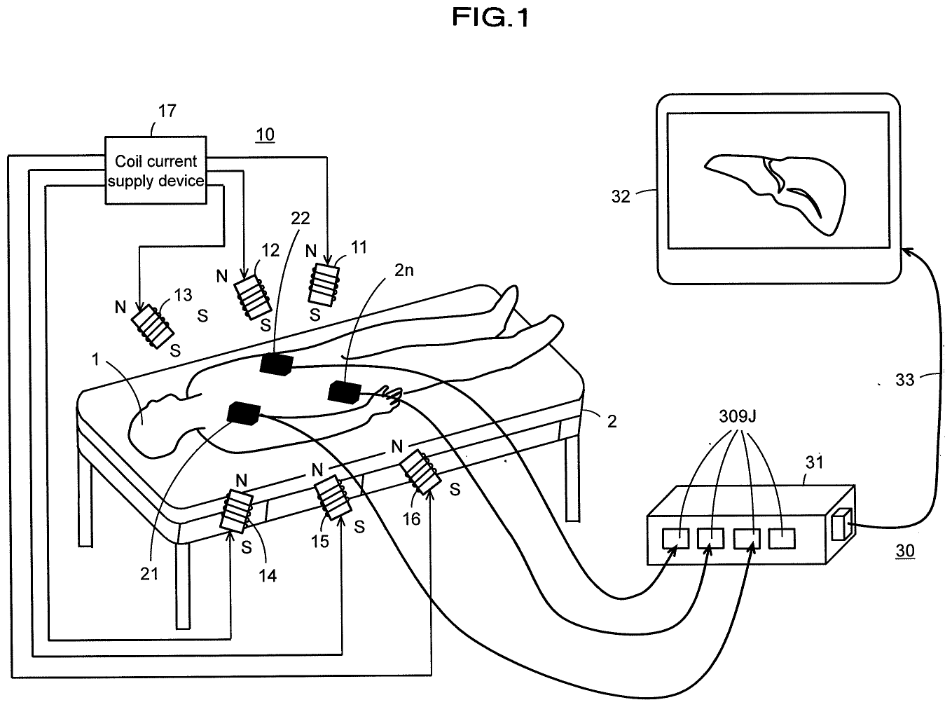

FIG. 1 is an overview showing an example of our surgical site monitoring system. The surgical site monitoring system comprises artificial magnetic field generation device 10, magnetic sensors 21 to 2n (n is an integer of 2 or more) and display control device 30.

Artificial magnetic field generation device 10 comprises coil current supply device 17 and electromagnets, such as 6 electromagnets 11 to 16 in FIG. 1 provided near operating table 2 on which patient 1 lies on his back. Electromagnets 11 to 16 comprise each coil wound on a magnetic core. Coil current supply device 17 supplies current for generating magnetic field to each coil of electromagnets 11 to 16.

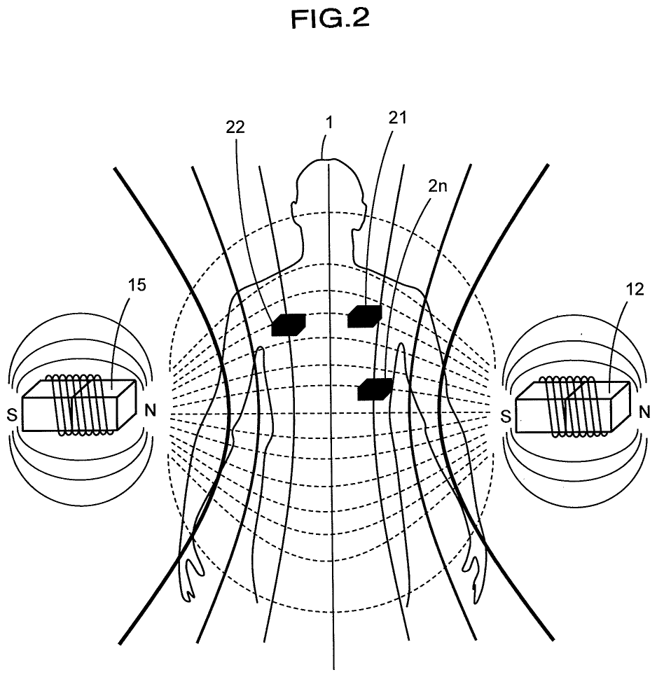

Artificial magnetic field generation device 10 generates artificial magnetic field so that a whole liver is within a 3-D space, which may be called “artificial magnetic field space, of the artificial magnetic field generated. To generate such artificial magnetic field space, at least two electromagnets may be provided at two positions interposing supine patient 1 on operating table 2 as shown in FIG. 2. FIG. 2 shows an example provided with electromagnets 12 and 15.

The value of pair of intensity and direction of magnetic field at each position is different to each other in the artificial magnetic field space. For example, FIG. 2 shows different directions of magnetic field along the solid lines while the intensity of magnetic field is the same along the solid lines. Further, FIG. 2 shows different intensity of magnetic field along the dotted lines while the direction is the same along the dotted lines. Therefore, when the value of pair of intensity and direction of magnetic field is detected at a position in an artificial magnetic field space, the position can be identified in the artificial magnetic field space.

Although it is important that the intensity and direction of magnetic field is detected by a magnetic sensor in the artificial magnetic field space accordingly, the artificial magnetic field space consisting of two electromagnets might have a position of which magnetic field intensity is too weak to detect in this example. So, six electromagnets 11 to 16 are provided so that the magnetic sensor can detect the intensity and direction of magnetic field with a high sensitivity at any position.

FIG. 1 shows six electromagnets 11 to 16, wherein three electromagnets 11 to 13 are provided on the left side of supine patient 1 on operating table 2 while three electromagnets 14 to 16 are provided on the right side of patient 1. Six electromagnets 11 to 16 are oriented so that the winding center line direction of coil wound around the magnetic core crosses the body of patient 1 while the end face in the winding center line direction of coil of magnetic core generating N pole or S pole faces to the body of patient 1. As shown in FIG. 2, each current is supplied from coil current supply device 17 to each electromagnet 11 to 16 so that magnetic poles different to each other are generated on the end face of each magnetic core facing to the body of patient 1 between three electromagnets 11 to 13 provided on the left side of patient 1 and three electromagnets 14 to 16 provided on the right side. FIGS. 1 and 2 show an example in which S pole is generated on the end face of magnetic core of electromagnets 11 to 13, which is provided on the left side of patient 1 as facing to the body of patient 1 while N pole is generated on the end face of magnetic core of electromagnets 14 to 16, which is provided on the right side of patient 1 as facing to the body of patient 1.

Although six electromagnets 11 to 16 may be provided in a 2-D plane, it is preferable that they are provided in a 3-D plane. It is possible that six electromagnets 11 to 16 are fixed to operating table 2, or are attached through separate attachments to peripheries of operating table 2. Besides, six electromagnets 11 to 16 are supposed to be fixed to operating table 2 after installation (especially during operation).

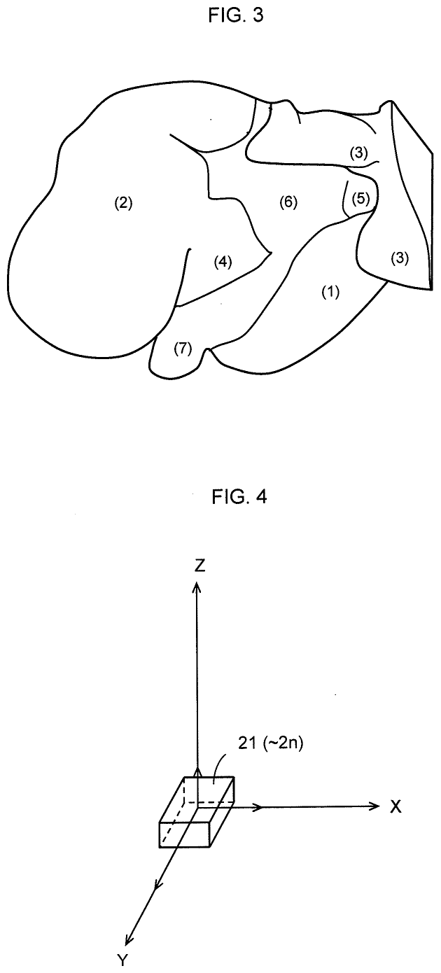

Magnetic sensors 21 to 2n are attached to a surgical site such as liver of patient 1 in advance of the operation. A predetermined number of magnetic sensors 21 are attached to predetermined places (positions) in the surgical site of patient 1. FIG. 3 is a schematic view of liver of human being, in which seven attachment places (indicated by numbers in parentheses) are predetermined so that the sensors are attached to the attachment places of the liver in advance of the operation in the same manner as endoscopic operation. Seven magnetic sensors are attached to the liver although FIG. 1 or the like only shows three magnetic sensors attached to the liver for the sake of simple drawing.

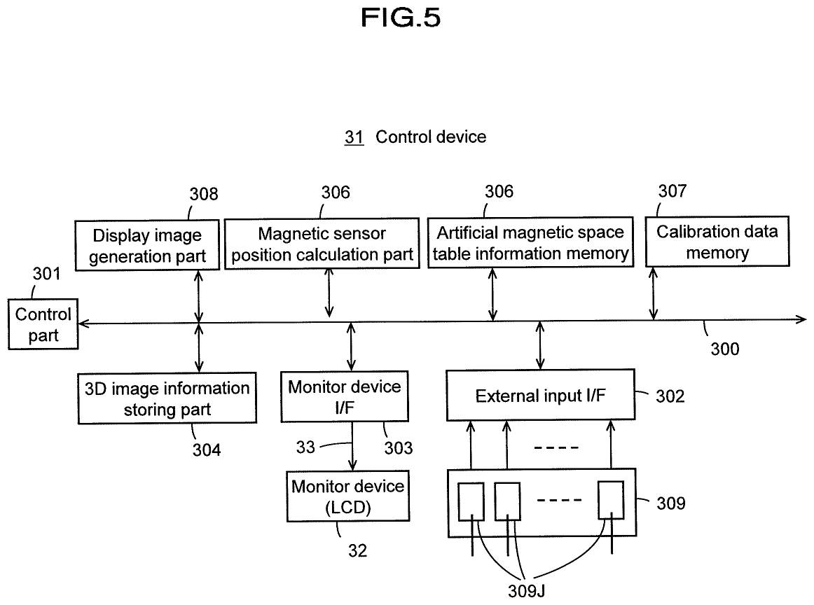

Magnetic sensors 21 to 2n have a compact size of about 1 mm for example, and output three axial output information for mutually perpendicular X-axis, Y-axis and Z-axis according to the detected intensity and direction of magnetic field as shown in FIG. 4. Magnetic sensors 21 to 2n are connected through connection cables to connector terminal 309 of control device 31 of display control device 30.

It is possible to confirm if magnetic sensors 21 to 2n are attached properly to the predetermined attachment places in the liver by X-ray photography, CT (Computerized Tomography) or the like.

Display control device 30 consists of control device 31 and monitor device 32 connected to control unit 31 through cable 33 in this example. Display control unit 30 consists of separate control unit 31 and monitor device 32 in this example, although it may consist of a device integrated by control unit 31 and monitor device 32.

Monitor device 32 is provided with a display screen of LCD (Liquid Crystal Display) or the like.

Control unit 31 configured like a computer device comprises a memory unit storing 3-D (3 Dimension) image data of the liver or the like and a control unit generating a display image to be displayed on the display screen of monitor device 32.

FIG. 5 is block diagram showing a hardware configuration example of control device 31. Control device 301 embedded with computer comprises system bus 300 connected to external input interface 302, monitor device interface (interface may be abbreviated as “I/F”) 303, 3-D image information memory unit 304, artificial magnetic field space table information memory 305, magnetic sensor position calculation unit 306, calibration data memory 307 and display image generation unit 308.

External input I/F 302 is connected to connector terminal unit 309 provided with connector jacks 309J. Connector plugs provided at the end of connection cables of magnetic sensors 21 to 2n are inserted into connector jacks 309J of input connector terminal unit 309 so that magnetic sensors 21 to 2n are electrically connected to control unit 31.

Monitor device 32 is connected through cable 33 with monitor device I/F 303 so that display image information generated by display image generation unit 308 is sent to monitor device 32 through this monitor device I/F 303.

3-D image information memory unit 304 preliminarily stores 3-D image data of surgical site such as liver or the like. The 3-D image data of liver is generated based on image data obtained by CT scan of liver of patient 1 before operation. The 3-D image data includes magnetic sensors 21 to 2n attached to the liver by performing the CT scan of liver provided with magnetic sensors 21 to 2n. Therefore, it is possible to confirm if magnetic sensors 21 to 2n have been attached properly to the predetermined places in the liver by displaying the 3-D image on the display screen.

In this example, positional information of magnetic sensors 21 to 2n on the 3-D image of the liver is obtained based on image data generated by CT scan of the liver or the like. The obtained positional information is stored in 3-D image information memory unit 304 according to information to identify magnetic sensors 21 to 2n.

It is necessary to coordinate each of magnetic sensors 21 to 2n attached to the liver as a surgical site of patient 1 and each of the positions of magnetic sensors 21 to 2n on the 3-D image of the liver as a surgical site of patient 1.

In this example, each of magnetic sensors 21 to 2n is identified by each of information of the connector number allocated to each of connector jacks 309J of input connector terminal unit 309. Each of magnetic sensors 21 to 2n is provided with each connector plug having information corresponding to the connector number or the like to identify an attachment place in the liver. Each of connector plugs at the end of cables of magnetic sensors 21 to 2n is connected to connector jack 309J having each corresponding number of input connector terminal unit 309 of control unit 31.

Thus, each of magnetism sensors 21 to 2n attached to the liver as a surgical site of patient 1 properly corresponds to each position of magnetic sensors 21 to 2n on the 3-D image of the liver as a surgical site of patient 1.

The coordination method is not limited specifically, and therefore various methods are available other than the above-described method in which each of magnetism sensors 21 to 2n attached to the liver as a surgical site of patient 1 properly corresponds to each position of magnetic sensors 21 to 2n on the 3-D image of the liver as a surgical site of patient 1.

For example, one of magnetic sensors 21 to 2n is attached to position (1) of the liver shown in FIG. 3, and then the connector jack at the end of cable for the magnetic sensor is connected to any one of connector jacks 309J of input connector terminal unit 309 of control unit 31. Control unit 31 displays prompts an input of the number of position on the 3-D image of magnetic sensor corresponding to the output information of magnetic sensor sent to connector jack 309 connected, as displaying an image with the prompt on the display screen of monitor device 32. For example, positional number (1) of magnetic sensor on the 3-D image is input by the installation worker of magnetic sensors. Then, control unit 31 stores identification information in the output signal of the magnetic sensor together with positional number (1) on the 3-D image. Other magnetic sensors are attached as well, so that control unit 31 stores coordination information between identification information in the output signal of each magnetic sensor and positional number on the 3-D image.

Accordingly, control unit 31 can recognize each attachment place of magnetic sensor on the 3-D image by referring to the stored coordination information according to the identification information in the output signal of the magnetic sensor input through input connector terminal unit 309.

Artificial magnetic field space table information memory 305 stores a correspondence table information between each position in artificial magnetic field space generated by artificial magnetic field generation device 10 and a pair of position and intensity and direction of magnetic field at the position. The correspondence table information may be generated according to actual value at each position in the artificial magnetic field space, or alternatively, be generated by calculating intensity and direction of magnetic field at each position in the artificial magnetic field space according to attachment places of electromagnets 11 to 16 constituting artificial magnetic field generation device 10, electric current applied to coils of electromagnets 11 to 16 or the like.

In this example, directions of coordinate axis such as X-axis, Y-axis and Z-axis are predetermined in 3-D artificial magnetic field space. For example, Z-axis is set perpendicular to the surface of operating table 2 on which patient 1 lies down, X-axis is set along the height direction of supine patient 1, and Y-axis is set perpendicular to the height direction of supine patient 1.

In this example, 3-D image information memory unit 304 stores 3-D image of which coordinate axes of X-axis, Y-axis and Z-axis are set along coordinate axes of X-axis, Y-axis and Z-axis in the artificial magnetic field space. Therefore, the monitor device 32 of which display screen is set perpendicular to Z-axis displays a liver viewed from the same direction as that of the surgeon's view of supine patient 1 on operating table 2 from the above.

Magnetic sensor position calculation unit 306 detects intensity and direction of magnetic field at the position of each magnetic sensor according to output information of each magnetic sensor 21 to 2n input through input connector terminal unit 309. Then, each corresponding position in the artificial magnetic field space is detected with reference to artificial magnetic field space table information memory 305 according to the pair of detected intensity and direction of magnetic field.

When each direction of X-axis, Y-axis and Z-axis shown in FIG. 4 of magnetic sensors 21 to 2n attached to the surgical site such as liver is set along each direction of X-axis, Y-axis and Z-axis in the artificial magnetic field space for obtaining correspondence table stored in artificial magnetic field space table information memory 305, the pair of intensity and direction of magnetic field detected according to output information of magnetic sensors 21 to 2n can be regarded as reference information of artificial magnetic field space table information memory 305.

However, each direction of X-axis, Y-axis and Z-axis of magnetic sensors 21 to 2n attached to liver of which surface is not flat is not in parallel with each direction of X-axis, Y-axis and Z-axis in the artificial magnetic field space usually. Accordingly, each of magnetic sensors 21 to 2n should be calibrated to deal with the difference between each direction of X-axis, Y-axis and Z-axis of magnetic sensors and each direction of X-axis, Y-axis and Z-axis in the artificial magnetic field space.

In this example, magnetic sensors 21 to 2n are calibrated to generate each calibration data for magnetic sensors 21 to 2n in advance of performing operation. Calibration data memory 307 stores the generated calibration data.

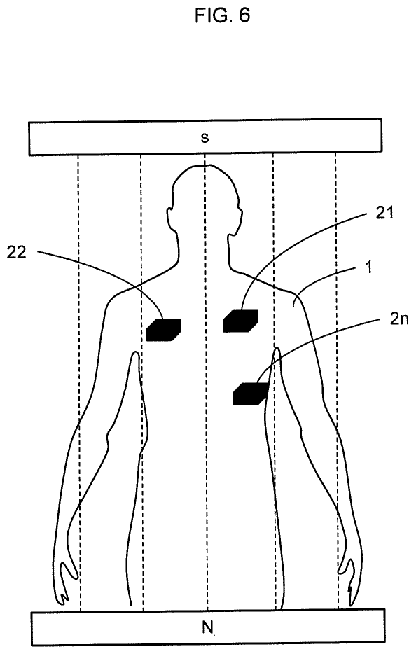

The calibration processing will be explained below. In this example, artificial magnetic field generation is suspended in advance of operation by suspending the supply of current from artificial magnetic field generation device 10 to coils of electromagnets 11 to 16. Then, geomagnetic field only is applied to supine patient 1 lying down on operating table 2 and having the liver attach magnetic sensors 21 to 2n. The geomagnetic field is formed as parallel magnetic field space in which intensity and direction of magnetic field are uniform as shown in FIG. 6 with dotted lines. In FIG. 6, patient 1 has a head on the south side and feet on the north side.

In such a condition, 3-axis output of the geomagnetic field corresponding to the attachment condition of liver of the patient is obtained from each of magnetic sensors 21 to 2n. Because each of thus obtained 3-axis output of magnetic sensors 21 to 2n the geomagnetic field corresponds to the difference between magnetic sensors 21 to 2n and the geomagnetic field, calibration data can be generated based on the known intensity and direction of geomagnetic field.

If the direction of geomagnetic field corresponded to Y-axis direction (direction toward feet from head of patient 1) corresponding to the geomagnetic field in the artificial magnetic field space generated by artificial magnetic field generation device 10, the difference to the artificial magnetic field space could be calibrated based on calibration data obtained from each 3-axis output of magnetic sensors 21 to 2n during the calibration processing.

However, the surface of operating table 2 is not flat practically while the direction toward feet from head of patient 1 cannot always be set to the north-south direction. In this example, the difference between the geomagnetic field direction and 3-axis direction of artificial magnetic field space generated by artificial magnetic field generation device 10 is obtained in advance, so that calibration data with respect to output information of magnetic sensors 21 to 2n for calibrating the difference to 3-axis direction of artificial magnetic field space is generated according to the obtained 3-axis output of magnetic sensors 21 to 2n. Then calibration data memory 307 stores the generated calibration data. The calibration data stored in calibration data memory 307 is calculated with respect to each of magnetic sensors 21 to 2n and is coordinated to each of identification information of magnetic sensors 21 to 2n.

In case that the geomagnetic field is hard to be detected because of operating room which may be shielded magnetically, the above-described calibration processing can be performed with artificial magnetic field space (parallel magnetic field space) generated so that intensity and direction of magnetic field are uniform like the geomagnetic field.

Magnetic sensor position calculation unit 306 detects intensity and direction of magnetic field at each position of magnetic sensors from output information of magnetic sensors 21 to 2n, and then the intensity and direction of the detected magnetic field is calibrated according to calibration data in calibration data memory 307 with respect to each of magnetic sensors 21 to 2n. Then each corresponding position in the artificial magnetic field space is detected according to the pair of intensity and direction of magnetic field calibrated with respect to each of magnetic sensors 21 to 2n with reference to artificial magnetic field space table information memory 305. Magnetic sensor position calculation unit 306 transmits each calculated positional information of transfers positional information of magnetic sensors 21 to 2n in the artificial magnetic field space to display image generation unit 308.

Display image generation unit 308 generates display image information to be displayed on the display screen of monitor device 32 so that 3-D image of the surgical site such as liver is drawn in the 3-D coordinate space of artificial magnetic field generated by artificial magnetic field generation device 10 according to 3-D image data stored in 3-D image information memory unit 304. Display image generation unit 308 generates display image information of the liver so that each position of attachment place of magnetic sensors 21 to 2n stored in 3-D image information memory unit 34 on 3-D image of the liver corresponds to each position of magnetic sensors 21 to 2n calculated by magnetic sensor position calculation unit 306.

Display image generation unit 308 sends generated 2-D display image information through monitor device I/F 303 to monitor device 32. Therefore, 3-D image (2-D display image) of liver of patient 1 displayed on the display screen of monitor device 32 corresponds to the state of the presence of the surgical site such as liver of supine patient 1 lying down on operation table 2 in the artificial magnetic field space.

Accordingly, the surgeon can perform an endoscopic operation properly and quickly by looking at a whole image of the surgical site such as liver displayed on the display screen of monitor 32.

In this example, output information of magnetic sensors 21 to 2n is always sent to control device 31 even in an operation so that magnetic sensor position calculation unit 306 of control device 31 can detect positions of magnetic sensors 21 to 2n. Magnetic sensor position calculation unit 306 always sends positional information of magnetic sensors 21 to 2n in the artificial magnetic field space to display image generation unit 308.

Display image generation unit 308 generates the display image information of the 3-D image of the liver according to positional information of magnetic sensors 21 to 2n sent from magnetic sensor position calculation unit 306 and continues supplying it to monitor device 32. Therefore, when the positions of magnetic sensors 21 to 2n move in the artificial magnetic field space by the motion of patient 1 or cutting the liver with a surgeon's knife, 3-D image of the liver displayed on the display screen of the monitor device 32 changes according to the movement. Therefore, the surgeon can advantageously recognize the change by looking at the image of the surgical site such as liver displayed on the display screen of monitor device 32, even when patient 1 moves to change the state of the presence of the surgical site such as liver in the artificial magnetic field space.

Although magnetic sensor position calculation unit 306 may always send calculated positional information of magnetic sensors 21 to 2n as described above, it can send positional information of magnetic sensors 21 to 2n moving in the artificial magnetic field space to display image generation unit 308 only when it is determined that any one of magnetic sensors 21 to 2n has moved over a distance greater than a predetermined threshold value. In such a case, display image generation unit 308 can continue to send the same display image information to monitor device 32 until the movement of the position of magnetic sensors 21 to 2n is detected. The predetermined threshold value to determine if any one of magnetic sensors 21 to 2n has moved should be set as a value corresponding to a moving distance detectable on the screen of monitor device 32.

### Modified Examples Based on the Above Described Examples

### <Magnetic Noise Reduction>

In the above-described examples, artificial magnetic field generation device 10 generates a fixed artificial magnetic field space. However, magnetic noise to negatively affect on output information of magnetic sensors 21 to 2n generally exists in the artificial magnetic field space in a room such as operating room. An example of the noise reduction method will be explained below.

In this example, different artificial magnetic field spaces including surgical sites are generated with switching for time sharing. The positions of magnetic sensors 21 to 2n in the artificial magnetic field space are detected according to output information of magnetic sensors 21 to 2n in the artificial magnetic field spaces. The obtained positions of magnetic sensors 21 to 2n are calculated in terms of position in a specific coordinate space, and then the converted positions of magnetic sensors 21 to 2n may be averaged.

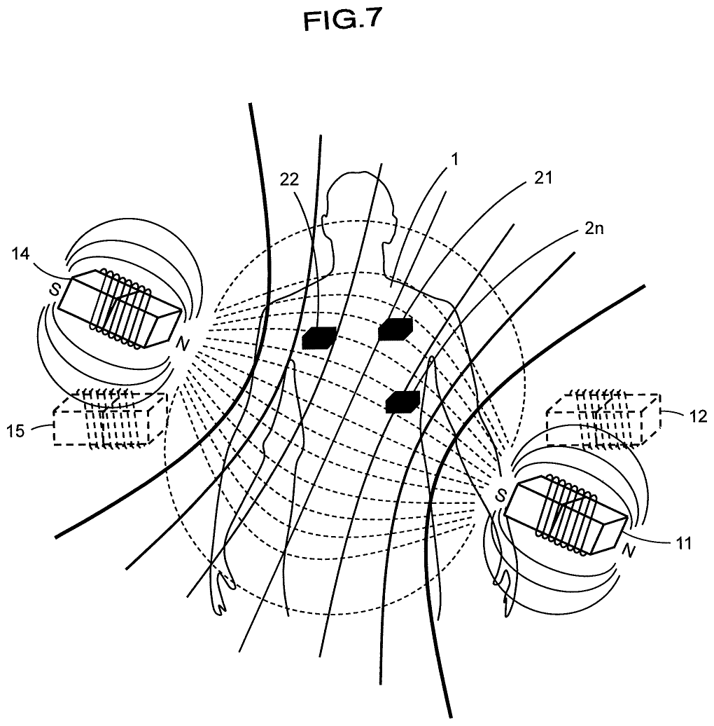

For example, when the artificial magnetic field space is formed by two electromagnets 12 and 15 as shown in FIG. 2, the same artificial magnetic field space as shown in FIG. 2 is generated by two electromagnets 12 and 15 indicated with dotted lines in FIG. 7 at the time of detecting positions of magnetic sensors 21 to 2n so that the positions of magnetic sensors 21 to 2n in the artificial magnetic field space are detected according to output information of magnetic sensors 21 to 2n in the artificial magnetic field space.

Next, electromagnets 12 and 15 are switched to electromagnets 11 and 14 to generate artificial magnetic field space shown in FIG. 7 with solid lines instead of FIG. 2. Then, the positions of magnetic sensors 21 to 2n in the artificial magnetic field space generated by electromagnets 11 and 14 are detected according to output information of magnetic sensors 21 and 2n in the artificial magnetic field space.

The positions of magnetic sensors 21 to 2n are detected as a position in a specific coordinate space. The specific coordinate space preferably improves the detection of positions of magnetic sensors 21 to 2n as a position in 3-D image of surgical site. The average of each position of magnetic sensors 21 to 2n in two artificial magnetic field spaces in the specific coordinate space is regarded as positions of magnetic sensors 21 to 2n to be obtained.

In a short time to switch a plurality of artificial magnetic field spaces, the positions of magnetic sensors 21 to 2n don't change in the surgical sites. When there exists no magnetic noise the positions of magnetic sensors 21 to 2n detected in terms of positions in the specific coordinate space are the same in different artificial magnetic field spaces. However, since the magnetic noise exists practically, the positions of magnetic sensors 21 to 2n in the two artificial magnetic field spaces calculated according to output information are not the same. The magnetic noise existing in the same manner between the two artificial magnetic field spaces can be halved by averaging the positions of each magnetic sensors 21 to 2n in the two artificial magnetic field space.

The above-described explanation is about a case that two artificial magnetic field spaces are generated to be switched for time sharing. When there are three or more artificial magnetic spaces, the magnetic noise could be reduced by a ratio of 1/m, where m means the number of artificial magnetic spaces. In FIG. 7, four pairs in total of electromagnets 12 and 15, electromagnets 12 and 14, electromagnets 11 and 14, electromagnets 11 and 15 can be switched for time sharing 7.

### <Improvement of Sensitivity Drop by the Position in the Artificial Magnetic Field Space>

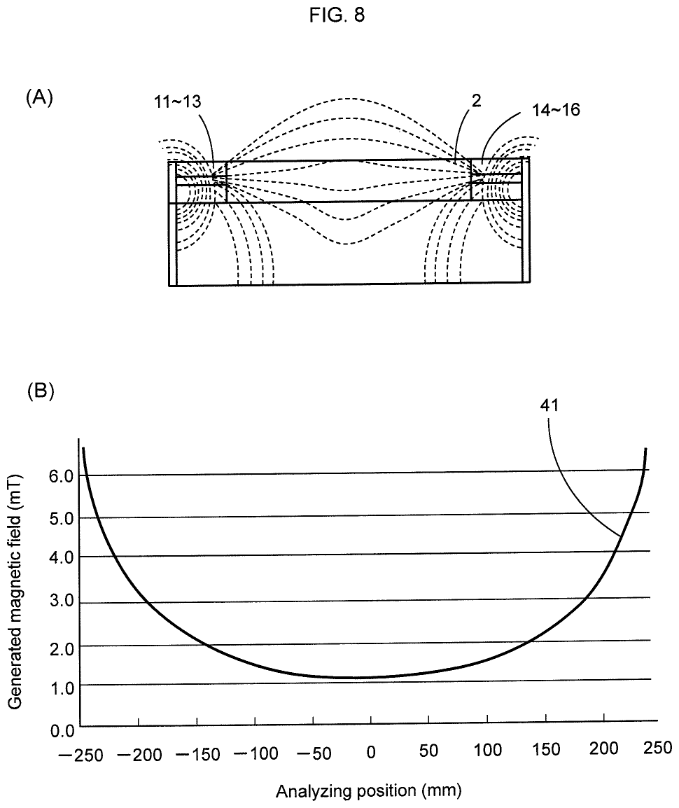

Because the patient is generally lying down on operating table 2 during operation, electromagnets 11 to 13 and 14 to 16 for generating artificial magnetic field space tend to be provided at both sides of operating table 2 as shown in FIG. 8 (A). Accordingly, magnetic field lines are as shown in FIG. 8 (A) while the relation between generated magnetic field and the position (the center of operating table) on the flat surface in the planar direction is as shown in FIG. 8 (A) and FIG. 8 (B) with solid line 41.

According to characteristics shown in FIG. 8 (B), intensity of generated magnetic field less changes at the surgical site of patient 1 regardless of positional change in the planar surface direction of operating table so that detectivity of positions of magnetic sensors 21 to 2n might be deteriorated.

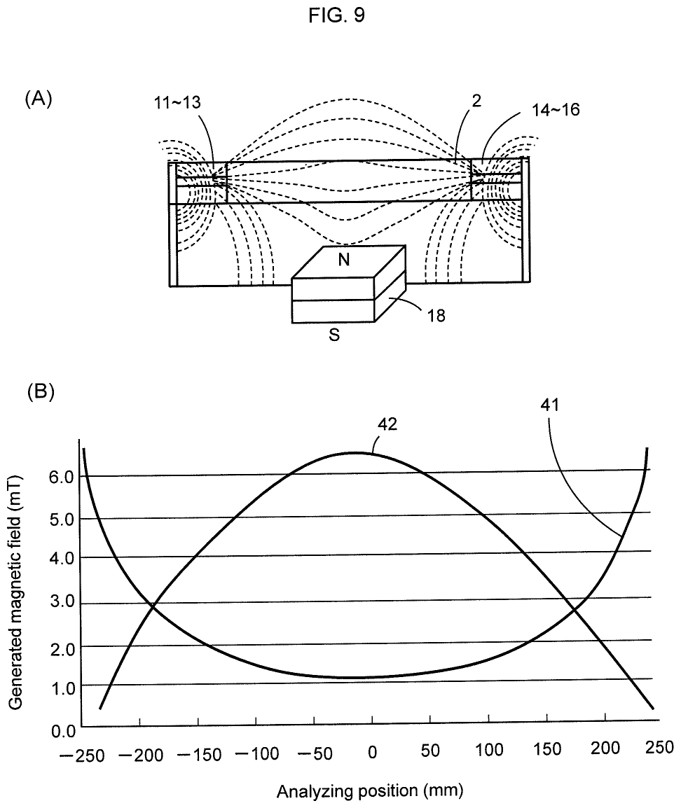

To improve the problem, external magnetic field generation means 18 such as electromagnet and permanent magnet is provided on the back side of supine patient on operating table 2 as shown in FIG. 9 (A). With this configuration, intensity of generated magnetic field changes as shown in FIG. 9 (B) with solid line 42 even around the center of operating table 2 in the planar surface direction of operating table so that detectivity of positions of magnetic sensors 21 to 2n can be prevented.

### Other Examples and Modified Example

The calibration processing is not limited specifically to that of the above-described examples, in which the calibration processing is performed with parallel magnetic field such as geomagnetic field to deal with the difference between each of magnetic sensors 21 to 2n and the artificial magnetic field space with respect to coordinate axes of X-axis, Y-axis and Z-axis.

For example, each of magnetic sensors 21 to 2n is provided with a gyro sensor (magnetic gyro sensor) to detect the difference (inclination) between coordinate axes of each of magnetic sensors 21 to 2n attached to surgical sites and coordinate axes of the artificial magnetic field space with respect to X-axis, Y-axis and Z-axis. For example, each of magnetic sensors 21 to 2n is provided with a gyro sensor attached or gyro sensor function while the coordinate axes of artificial magnetic field space such as X-axis direction and Y-axis direction are aligned to the body side direction and head direction of supine patient lying on operating table 2 of which surface is in parallel with the coordinate axes and Z-axis direction is perpendicular to the surface.

With such a configuration, the difference (inclination to each coordinate axis) between coordinate axes of each of magnetic sensors 21 to 2n attached to surgical sites and coordinate axes of the artificial magnetic field space with respect to X-axis, Y-axis and Z-axis can be detected by each gyro sensor to generate calibration data to be stored in calibration data memory 307 according to the detected difference (inclination to each coordinate axis). Each output information of magnetic sensors 21 to 2n is calibrated according to calibration data stored in calibration data memory 307 in the same manner as the above-described examples.

When magnetic sensors are calibrated with a gyro sensor like this example, the surgeon can use a gyro sensor embedded with magnetic sensors attached to surgical implement for endoscopic operation, so that the position of the surgical implement corresponding to the surgical site can be displayed on the display screen of monitor device 32.

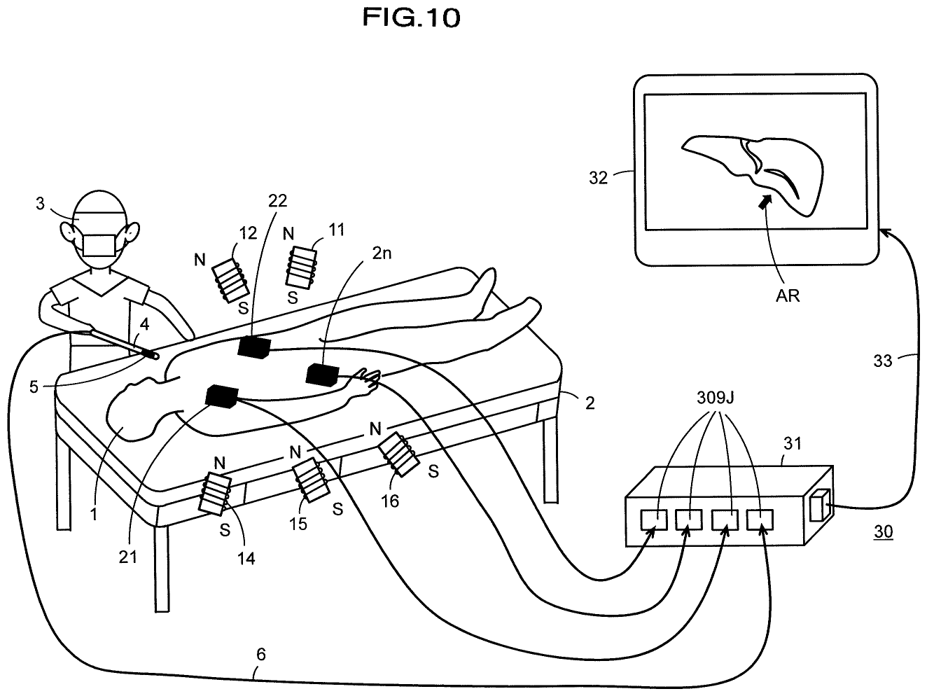

FIG. 10 shows an example in which a gyro sensor is embedded with magnetic sensor 5 provided on the tip of surgical implement 4 for endoscopic operation that surgeon 3 holds. Magnetic sensor 5 is connected through connection cable 6 to one of connector jacks 309J of input connector terminal unit 309 of control unit 31 of display control unit 30.

In FIG. 10, control unit 31 calibrates output information of magnetic sensors 21 to 2n according to calibration data stored in calibration data memory 307 to detect positions of magnetic sensors 21 to 2n, so that 3-D image of surgical sites generated based on the detected positions are displayed on the display screen of monitor device 32. The calibration date stored in calibration data memory 307 may be obtained with the above-described parallel magnetic field, or alternatively, may be obtained according to the difference (inclination to each coordinate axis) from the gyro sensor.

Control unit 31 calibrates output information of magnetic sensor 5 attached to surgical implement 4 connected to one of connector jacks 309J of input connector terminal unit 309 according to gyro sensor output included in the output information to detect a position of magnetic sensor 5 in the artificial magnetic field space, so that the tip position of surgical implement 4 with respect to 3-D image of surgical site is displayed on the display screen of monitor device 32. FIG. 10 indicates arrow AR as a position of surgical implement 4.

Although the magnetic sensor is connected to control device 31 of display control device 30 through a cable in the above-described examples, it can be connected wirelessly when each magnetic sensor is provided with wireless communication function while control device 31 is provided with wireless function capable of receiving output information from the magnetic sensor. In such wireless communication, each magnetic sensor transmits wireless signal including identification information to identify the sensor itself. Control device 31 stores identification information of each magnetic sensor in coordination with attachment position in the surgical site in the same manner as the above-described examples.

### EXPLANATION OF SYMBOLS

1: patient
2: operating table
3: surgeon
4: surgical implement
5: magnetic sensor
10: artificial magnetic field generation device
11-16: electromagnet
21-2n: magnetic sensor
30: display control device
31: control device
32: monitor device
33: connection cable
301: control unit
304: 3-D image information memory unit
305: artificial magnetic field space table information memory
306: magnetic sensor position calculation unit
307: calibration data memory
308: display image generation unit
309: input connector terminal unit

1. A system for monitoring a surgical site, comprising:
an artificial magnetic field generation device to generate a 3-D artificial magnetic field space in which a surgical site of a patient is disposed;
a plurality of magnetic sensors attached to attachment places in the surgical site of the patient to detect a position of the attachment place in the artificial magnetic field; and
a display control device to obtain output information output from the magnetic sensors as identifiable to each other,
wherein the display control unit comprises:
a monitor unit provided with a display screen;
a memory unit to store a 3-D image data of the surgical site and a positional information of the attachment place with respect to a 3-D image of the surgical site; and
a control unit to display the 3-D image, which corresponds to a present condition of the surgical site within the artificial magnetic field space, on the display screen of the monitor unit based on the 3-D image data stored in the memory unit, the positional information of the attachment place of the magnetic sensor with respect to the 3-D image of the surgical site, and the output information output from the plurality of magnetic sensors.

2. The system for monitoring a surgical site according to claim 1, wherein the control unit of the display control unit changes the 3-D image of the surgical site displayed on the display screen according to a change of the positional information of the attachment place of the magnetic sensor in the artificial magnetic field space.

3. The system for monitoring a surgical site according to claim 1, wherein the surgical site is a minimally invasive surgical site.

4. The system for monitoring a surgical site according to claim 1, wherein the surgical site is an organ.

5. The system for monitoring a surgical site according to claim 1, wherein the display control unit provided with a plurality of terminals for receiving the output information through a connection cable identifies the terminal which has received the output information of the magnetic sensors to obtain output information output from the magnetic sensors as identifiable to each other.

6. The system for monitoring a surgical site according to claim 1, wherein the display control unit is wirelessly connected to the magnetic sensors which send an identification information for identifying the sensors themselves added to the output information to the display control unit to obtain the output information of the magnetic sensors as identifiable to each other.

7. The system for monitoring a surgical site according to claim 1, wherein the display control unit has a correspondence table coordinating the position of the artificial magnetic field space with the intensity and the direction of a magnetic field at the position, the control unit detecting a position of the magnetic sensor in the artificial magnetic field space according to the correspondence table with reference to the intensity and the direction of the magnetic field detected according the output information of the magnetic sensors.

8. The system for monitoring a surgical site according to claim 1, wherein the display unit has a calibration function to obtain a calibration data to calibrate a difference between the magnetic sensors and the artificial magnetic field space with respect to coordinate axes, the control unit calibrates the output information of the magnetic sensors within the artificial magnetic space according to the calibration data.

9. The system for monitoring a surgical site according to claim 8, wherein the calibration data is obtained by applying a parallel magnetic field having a uniform direction to the magnetic sensors while the artificial magnetic field generation device stops generating the artificial magnetic field.

10. The system for monitoring a surgical site according to claim 9, wherein the parallel magnetic field is a geomagnetic field.

11. The system for monitoring a surgical site according to claim 9, wherein the parallel magnetic field is a geomagnetic field.

12. The system for monitoring a surgical site according to claim 8, wherein the calibration data is obtained by detecting a difference from the coordinate axes of the magnetic sensors attached to the surgical sites of the patient lying on an operation table with a magnetic gyro sensor.

13. The system for monitoring a surgical site according to claim 1, wherein the artificial magnetic field generation device generates a plurality of artificial magnetic field spaces in which the intensity and the direction of the magnetic field at the surgical sites are different to each other, the display control device calculating a position of the magnetic sensor in the artificial magnetic space according to the output information of the magnetic sensors in the artificial magnetic field spaces.

14. The system for monitoring a surgical site according to claim 1, further comprising an external magnetic field applying means to apply a magnetic field from an outside of the patient to a partial space provided with the magnetic sensors in the artificial magnetic field space.

15. The system for monitoring a surgical site according to claim 1, wherein the display control device connected to a magnetic sensor attached to a tip of a surgical implement displays a position of the tip of the surgical implement with respect to the 3-D image of the surgical site according to the output information of magnetic sensor attached to the tip of the surgical implement, the output information of magnetic sensor attached to the tip of the surgical implement being identifiable from the output information of magnetic sensors attached to the surgical sites.

16. A method for monitoring a surgical site, comprising:
an artificial magnetic field generation step to generate an artificial magnetic field space so that surgical sites of a patient of which a plurality of attachment places are attached to magnetic sensors in the artificial magnetic field space; and
a first display control step to display a 3-D image corresponding to a present condition of the surgical site within the artificial magnetic field space on a display screen of a monitor unit, based on a 3-D image data stored in a memory unit, a positional information of the attachment place of the magnetic sensor stored with respect to the 3-D image of the surgical site and an output information of the magnetic sensors being identifiable from each other.

17. The method for monitoring a surgical site according to claim 16, further comprising a second display control step to change the 3-D image of the surgical site displayed on the display screen according to a change of the positional information of the attachment place of the magnetic sensor in the artificial magnetic field space.

18. The method for monitoring a surgical site according to claim 16, further comprising a calibration step in advance of the artificial magnetic field generation step, wherein a calibration data is obtained to calibrate a difference between the magnetic sensors and the artificial magnetic field space with respect to coordinate axes, and then the output information of the magnetic sensors within the artificial magnetic space is calibrated according to the calibration data.

19. The method for monitoring a surgical site according to claim 16, further comprising a third display control step to display a position of a tip of a surgical implement with respect to the 3-D image of the surgical site according to the output information of magnetic sensor attached to the tip of the surgical implement, wherein the output information of magnetic sensor attached to the tip of the surgical implement is identifiable from the output information of magnetic sensors attached to the surgical sites.

20. A non-transitory medium including a computer program for executing a display control step, wherein a computer constitutes a surgical site monitoring system comprising an artificial magnetic field generation device to generate a 3-D artificial magnetic field space in which a surgical site of a patient is disposed; a plurality of magnetic sensors attached to attachment places in the surgical site of the patient to detect a position of the attachment place in the artificial magnetic field; and a display control device to obtain output information output from the magnetic sensors as identifiable to each other,
the display control step comprising a first display control step to display a 3-D image corresponding to a present condition of the surgical site within the artificial magnetic field space on a display screen of a monitor unit, based on the output information of one or more magnetic sensors and a 3-D image data of the surgical site stored together with a positional information of the attachment place with respect to the 3-D image of the surgical site in a memory unit.

21. The non-transitory medium including a computer program according to claim 20, wherein the display control step further comprises a second display control step to change the 3-D image of the surgical site displayed on the display screen according to a change of the positional information of the attachment place of the magnetic sensor in the artificial magnetic field space.
